(19) United States
(12) Patent Application Publication
(10) Pub. No.: US 2025/0041291 A1
(43) Pub. Date: Feb. 6, 2025

(54) COMPOSITIONS AND METHODS FOR TREATING CANCER

(71) Applicants: Emmaus Life Sciences, Inc, Torrance, CA (US); Kainos Medicine, Inc., Seongnam-si (KR)

(72) Inventors: Joan Oliva Vilana, Lakewood, CA (US); Yutaka Niihara, Rolling Hills Estates, CA (US)

(21) Appl. No.: 18/717,260

(22) PCT Filed: Apr. 6, 2022
(86) PCT No.: PCT/US2022/023632

§ 371 Date: Jun. 6, 2024

Related U.S. Application Data
(60) Provisional application No. 63/286,427, filed on Dec. 6, 2021.

(51) Int. Cl. A61K 31/496; A61K 31/502; A61K 31/519

(52) U.S. Cl. CPC A61K 31/496; A61K 31/502; A61K 31/519

(57) ABSTRACT

The present disclosure relates generally to methods for treating cancer, in particular hematologic cancers, with an N-(1H-imidazol-2-yl)benzamide compound of a salt thereof, optionally in combination with a PARP inhibitor or a BTK inhibitor. An example N-(1H-imidazol-2-yl)benzamide compound is 3-(1-methyl-1H-pyrazol-4-yl)-N-(4-(4-(4-methylpiperazin-1-yl)-4-oxobut yl)-1-phenyl-1H-imidazol-2-yl)benzamide, or a salt of hydrochloride, citrate, fumarate, (2R,3R)-2,3-dihydroxysuccinate, or succinate.

### CROSS REFERENCE TO RELATED APPLICATIONS

This application claims priority under 35 U.S.C. § 119(e) to U.S. Provisional Application No. 63/286,427, filed Dec. 6, 2021, which is hereby incorporated by reference in its entirety.

### BACKGROUND

Despite the availability of many treatment options, cancer remains one of the primary causes of mortality. Certain cancers may be more resistant to treatments or susceptible to recurrence due to their genetic compositions such as mutations.

Fms related receptor tyrosine kinase 3 (FLT3) is a proto-oncogene involved in important steps of hematopoiesis. FLT3 mutations have been associated with the clinical prognosis, treatment and survival of patients. The most common form of FLT3 mutation is an internal tandem duplication (ITD) that promotes ligand-independent auto-phosphorylation and constitutive activation of the receptor. FLT3-ITD has been strongly associated with a poor prognosis, leukocytosis, high blast counts, increased risk of relapse and shorter overall survival.

Likewise, the myeloid differentiation factor 88 (MyD88) gene is a driver gene found in hematologic B-cell malignancies. A missense mutation (L265P) changing leucine at position 265 to proline in MYD88 is found in ˜90% of Waldenström macroglobulinemia (WM) cases and in significant portions of activated B-cell diffuse large B-cell lymphomas and IgM monoclonal gammopathy of undetermined significance.

WM is a non-Hodgkin lymphoma, often associated with production of monoclonal IgM in a large amount. The increased level of IgM leads to the increased level of blood viscosity, potentially causing spontaneous bleeding, headaches, vertigo and could lead to stroke and coma. WM is a rare disease, affecting around 3 cases per million per year in the USA. Different chromosomal abnormalities can be cause of WM, but the most common mutation detected in WM is the L265P mutation of Myd88. While today there are many treatment options to manage WM, there isn't a single effective enough treatment for WM that are widely used.

### SUMMARY

The present disclosure made the unexpected discovery that Compound A, having a chemical name of 3-(1-methyl-1H-pyrazol-4-yl)-N-(4-(4-(4-methylpiperazin-1-yl)-4-oxobutyl)-1-phenyl-1H-imidazol-2-yl)benzamide, can effectively induce apoptosis of leukemia cells with the FLT3-ITD mutation, or WM cells with the Myd88 L265P mutation. Also surprisingly, when used along with a poly ADP ribose polymerase (PARP) inhibitor or a Bruton's tyrosine kinase (BTK) inhibitor, synergistic anti-cancer effects were observed.

Accordingly, one embodiment of the present disclosure provides a method of treating cancer in a patient, comprising administering to the patient a compound of the structure of

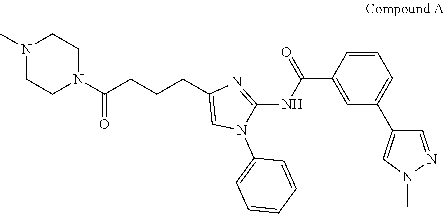 [SMILES: CN1CCN(C(=O)CCCc2cn(-c3ccccc3)c(NC(=O)c3cccc(-c4cnn(C)c4)c3)n2)CC1]

or a salt thereof.

In some embodiments, the salt is selected from the group consisting of hydrochloride salt, citrate salt, fumarate salt, (2R,3R)-2,3-dihydroxysuccinate salt, and succinate salt.

In some embodiments, the cancer is a hematologic cancer. In some embodiments, the hematological cancer is selected from the group consisting of acute lymphocytic leukemia (ALL), acute myeloid leukemia (AML), chronic lymphocytic leukemia (CLL), chronic myeloid leukemia (CML), prolymphocytic leukemia (PLL), large granular lymphocytic (LGL) leukemia, hairy cell leukemia (HCL), and myelodysplastic syndrome (MDS).

In some embodiments, the hematologic cancer is AML. In some embodiments, the patient has a fms related receptor tyrosine kinase 3 (FLT3) mutation. In some embodiments, the FLT3 mutation is the internal tandem duplicate mutation (FLT3-ITD).

In some embodiments, the hematological cancer is selected from the group consisting of Hodgkin's lymphoma, diffuse large B-cell lymphoma (DLBCL), mantle cell lymphoma (MCL), lymphoblastic lymphoma, Burkitt lymphoma (BL), primary mediastinal (thymic) large B-cell lymphoma (PMBCL), transformed follicular and transformed mucosa-associated lymphoid tissue (MALT) lymphoma, high-grade B-cell lymphoma with double or triple hits (HBL), primary cutaneous DLBCL of the leg, primary DLBCL of the central nervous system, primary central nervous system (CNS) lymphoma, acquired immunodeficiency syndrome (AIDS)—associated lymphoma, follicular lymphoma (FL), marginal zone lymphoma (MZL), chronic lymphocytic leukemia/small-cell lymphocytic lymphoma (CLL/SLL), gastric mucosa-associated lymphoid tissue (MALT) lymphoma, lymphoplasmacytic lymphoma, Waldenström macroglobulinemia (WM), nodal marginal zone lymphoma (NMZL), splenic marginal zone lymphoma (SMZL), peripheral T-cell lymphoma (PTCL), systemic anaplastic large-cell lymphoma (ALCL), lymphoblastic lymphoma, hepatosplenic gamma/delta T-cell lymphoma, subcutaneous panniculitis-like T-cell lymphoma (SPTCL), enteropathy-type intestinal T-cell lymphoma, primary cutaneous anaplastic large-cell lymphoma, angioimmunoblastic T-cell lymphoma (AITL), cutaneous T-cell lymphoma (CTCL), mycosis fungoides (MF), Sézary syndrome (SS), adult T-cell leukemia/lymphoma, and extranodal NK/T-cell lymphoma (ENK/TCL).

In some embodiments, the hematologic cancer is Waldenström macroglobulinemia (WM). In some embodiments, the patient has a L265P mutation in the myeloid differentiation factor 88 (MyD88) gene.

In some embodiments, the hematologic cancer is multiple myeloma.

In some embodiments, the hematologic cancer is selected from the group consisting of polycythemia vera, myelofibrosis, thrombocythemia, chronic neutrophilic leukemia, and eosinophilia.

In some embodiments, the cancer is a solid tumor. In some embodiments, the cancer is selected from the group consisting of bladder cancer, liver cancer, colon cancer, rectal cancer, endometrial cancer, pancreatic cancer, small cell lung cancer, non-small cell lung cancer, breast cancer, urethral cancer, head and neck cancer, gastrointestinal cancer, stomach cancer, esophageal cancer, ovarian cancer, renal cancer, melanoma, prostate cancer and thyroid cancer.

In some embodiments, the cancer is relapsed and refractory.

In some embodiments, the method further comprises administering to the patient a poly ADP ribose polymerase (PARP) inhibitor. In some embodiments, the PARP inhibitor is selected from the group consisting of olaparib, rucaparib, niraparib, talazoparib, veliparib, pamiparib, and iniparib. In some embodiments, the PARP inhibitor is olaparib.

In some embodiments, the method further comprises administering to the patient a Bruton's tyrosine kinase (BTK) inhibitor. In some embodiments, the BTK inhibitor is selected from the group consisting of ibrutinib, acalabrutinib, zanubrutinib, tirabrutinib, tolebrutinib, evobrutinib, fenebrutinib, pirtobrutinib, and spebrutinib. In some embodiments, the BTK inhibitor is ibrutinib.

Also provided, in one embodiment, is a composition comprising a compound of the structure of

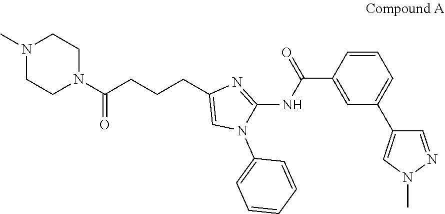 [SMILES: CN1CCN(C(=O)CCCc2cn(-c3ccccc3)c(NC(=O)c3cccc(-c4cnn(C)c4)c3)n2)CC1]

or a salt thereof and a PARP inhibitor or a BTK inhibitor.

In some embodiments, the salt is selected from the group consisting of hydrochloride salt, citrate salt, fumarate salt, (2R,3R)-2,3-dihydroxysuccinate salt, and succinate salt. In some embodiments, the PARP inhibitor is selected from the group consisting of olaparib, rucaparib, niraparib, talazoparib, veliparib, pamiparib, and iniparib. In some embodiments, the PARP inhibitor is olaparib. In some embodiments, the BTK inhibitor is selected from the group consisting of ibrutinib, acalabrutinib, zanubrutinib, tirabrutinib, tolebrutinib, evobrutinib, fenebrutinib, pirtobrutinib, and spebrutinib. In some embodiments, the BTK inhibitor is ibrutinib.

### BRIEF DESCRIPTION OF THE DRAWINGS

FIG. 1A-B show the effects of various compounds on proliferation and apoptosis of human adipose stromal cells (ASC) cells at 25% FBS.

FIG. 2A-B show the effects of various compounds on proliferation and apoptosis of human fibroblast primary cells at 25% FBS.

FIG. 3A-B show the effects of various compounds on proliferation and apoptosis of MOLM-13 cells (adult acute myeloid leukemia) at 2% FBS.

FIG. 4A-B show the effects of various compounds on proliferation and apoptosis of MOLM-13 cells at 10% FBS.

FIG. 5A-B show the effects of various compounds on proliferation and apoptosis of MOLM-13 cells at 25% FBS.

FIG. 6A-B show the effects of various compounds on proliferation and apoptosis of MWCL-1 cells (adult Waldenström macroglobulinemia cells) at 10% FBS.

FIG. 7A-B show the effects of various compounds on proliferation and apoptosis of MWCL-1 cells at 25% FBS.

FIG. 8A-B show the effects of various compounds on proliferation and apoptosis of MWCL-1 cells at 2% FBS.

FIG. 9 shows the effects of various compounds on apoptosis of MWCL-1 cells at 10% FBS.

FIG. 10A-B show the effects of various compounds on proliferation and apoptosis of MWCL-1 cells at 25% FBS.

FIG. 11A-B show the effects of various compounds on proliferation and apoptosis of MWCL-1 cells at 25% FBS.

FIG. 12A-B show the effects of various compounds on proliferation and apoptosis of MWCL-1 cells at 25% FBS.

### DETAILED DESCRIPTION

### Definitions

The following description sets forth exemplary embodiments of the present technology. It should be recognized, however, that such description is not intended as a limitation on the scope of the present disclosure but is instead provided as a description of exemplary embodiments.

As used in the present specification, the following words, phrases and symbols are generally intended to have the meanings as set forth below, except to the extent that the context in which they are used indicates otherwise.

A dash (“−”) that is not between two letters or symbols is used to indicate a point of attachment for a substituent. For example, —C(O)NH₂ is attached through the carbon atom. A dash at the front or end of a chemical group is a matter of convenience; chemical groups may be depicted with or without one or more dashes without losing their ordinary meaning. A wavy line drawn through a line in a structure indicates a point of attachment of a group. Unless chemically or structurally required, no directionality is indicated or implied by the order in which a chemical group is written or named.

Provided are also pharmaceutically acceptable salts, hydrates, solvates, tautomeric forms, polymorphs, and prodrugs of the compounds described herein. “Pharmaceutically acceptable” or “physiologically acceptable” refer to compounds, salts, compositions, dosage forms and other materials which are useful in preparing a pharmaceutical composition that is suitable for veterinary or human pharmaceutical use.

As used herein, “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

“Treatment” or “treating” is an approach for obtaining beneficial or desired results including clinical results. Beneficial or desired clinical results may include one or more of the following: a) inhibiting the disease or condition (e.g., decreasing one or more symptoms resulting from the disease or condition, and/or diminishing the extent of the disease or condition); b) slowing or arresting the development of one or more clinical symptoms associated with the disease or condition (e.g., stabilizing the disease or condition, preventing or delaying the worsening or progression of the disease or condition, and/or preventing or delaying the spread (e.g., metastasis) of the disease or condition); and/or c) relieving the disease, that is, causing the regression of clinical symptoms (e.g., ameliorating the disease state, providing partial or total remission of the disease or condition, enhancing effect of another medication, delaying the progression of the disease, increasing the quality of life, and/or prolonging survival.

“Subject” or “patient” refers to an animal, such as a mammal (including a human), that has been or will be the object of treatment, observation or experiment. The methods described herein may be useful in human therapy and/or veterinary applications. In some embodiments, the subject is a mammal. In one embodiment, the subject is a human.

The term “therapeutically effective amount” or “effective amount” of a compound described herein or a pharmaceutically acceptable salt, tautomer, stereoisomer, mixture of stereoisomers, prodrug, or deuterated analog thereof means an amount sufficient to effect treatment when administered to a subject, to provide a therapeutic benefit such as amelioration of symptoms or slowing of disease progression. For example, a therapeutically effective amount may be an amount sufficient to decrease a symptom of a disease or condition. The therapeutically effective amount may vary depending on the subject, and disease or condition being treated, the weight and age of the subject, the severity of the disease or condition, and the manner of administering, which can readily be determined by one or ordinary skill in the art.

### Cancer Treatments

The present disclosure, in various embodiments, provides compositions and methods for treating cancer, including hematologic cancers and solid tumors. The accompanying experimental examples tested the efficacy of a new compound, 3-(1-methyl-1H-pyrazol-4-yl)-N-(4-(4-(4-methylpiperazin-1-yl)-4-oxobutyl)-1-phenyl-1H-imidazol-2-yl)benzamide (Compound A), for its effect in inhibiting the growth and promoting apoptosis of various cancer cells. At effectively concentrations, Compound A was able to activate caspase 3/7 activities, leading to decreases of cancer cell numbers. Surprisingly, Compound A did not induce apoptosis of normal cells, including human adipose stromal cells (ASC) and human fibroblast primary cells. Accordingly, the data have demonstrated the activity of Compound A in treating cancers.

Another surprising discovery is that when used with a poly ADP ribose polymerase (PARP) inhibitor (e.g., olaparib) or a Bruton's tyrosine kinase (BTK) inhibitor (e.g., ibrutinib), Compound A synergized with this second agent in inducing cancer cell apoptosis, leading to more pronounced cancer inhibition.

In accordance with one embodiment of the present disclosure, provided is a method for treating cancer. In some embodiments, the method entails administering to a cancer patient Compound A or a salt thereof.

Compound A, with the chemical name of 3-(1-methyl-1H-pyrazol-4-yl)-N-(4-(4-(4-methylpiperazin-1-yl)-4-oxobutyl)-1-phenyl-1H-imidazol-2-yl)benzamide, has the following structure:

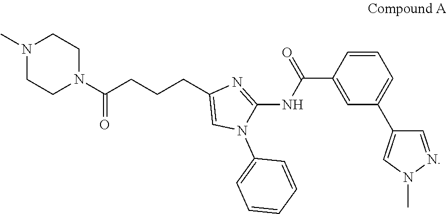 [SMILES: CN1CCN(C(=O)CCCc2cn(-c3ccccc3)c(NC(=O)c3cccc(-c4cnn(C)c4)c3)n2)CC1]

Compound A is an N-(1H-imidazol-2-yl)benzamide which has been described in PCT patent application No. PCT/KR2020/013397 as compound 143. Various salts of Compound A are also described, including a hydrochloride salt (compound 129), a citrate salt (compound 133), a fumarate salt (compound 134), a (2R,3R)-2,3-dihydroxysuccinate salt (compound 135), and a succinate salt (compound 136). The synthesis of Compound A is provided in Example 139. Syntheses of the salts are also described in the PCT application.

In some embodiments, the cancer being treated is a hematologic cancer. Non-limiting examples of hematologic cancers include leukemia, lymphoma, myeloma (e.g., multiple myeloma), and relatively rare myeloproliferative disorders.

Examples of leukemias include, without limitation, acute lymphocytic leukemia (ALL), acute myeloid leukemia (AML, also known as acute myelogenous leukemia, acute myeloblastic leukemia, acute granulocytic leukemia or acute nonlymphocytic leukemia), chronic lymphocytic leukemia (CLL), chronic myeloid leukemia (CML, also known as chronic myelogenous leukemia), prolymphocytic leukemia (PLL), large granular lymphocytic (LGL) leukemia, hairy cell leukemia (HCL), and myelodysplastic syndrome (MDS, also known as preleukemia).

Lymphoma may be Hodgkin's lymphoma or non-Hodgkin's lymphoma (NHL). There are more than 60 specific types of NHL, including many under the category of mature B-cell lymphoma or mature T-cell and NK-cell lymphoma. Example mature B-cell lymphomas include, without limitation:
Diffuse large B-cell lymphoma (DLBCL),
Mantle cell lymphoma (MCL),
Lymphoblastic lymphoma,
Burkitt lymphoma (BL),
Primary mediastinal (thymic) large B-cell lymphoma (PMBCL),
Transformed follicular and transformed mucosa-associated lymphoid tissue (MALT) lymphomas,
High-grade B-cell lymphoma with double or triple hits (HBL),
Primary cutaneous DLBCL, leg type,
Primary DLBCL of the central nervous system,
Primary central nervous system (CNS) lymphoma,
Acquired immunodeficiency syndrome (AIDS)—associated lymphoma,
Follicular lymphoma (FL),
Marginal zone lymphoma (MZL),
Chronic lymphocytic leukemia/small-cell lymphocytic lymphoma (CLL/SLL),
Gastric mucosa-associated lymphoid tissue (MALT) lymphoma,
Lymphoplasmacytic lymphoma,
Waldenström macroglobulinemia (WM),
Nodal marginal zone lymphoma (NMZL), and
Splenic marginal zone lymphoma (SMZL).

Example mature T-cell and NK-cell lymphomas include, without limitation:
Peripheral T-cell lymphoma (PTCL),
Systemic anaplastic large-cell lymphoma (ALCL),
Lymphoblastic lymphoma,
Hepatosplenic gamma/delta T-cell lymphoma,
Subcutaneous panniculitis-like T-cell lymphoma (SPTCL),
Enteropathy-type intestinal T-cell lymphoma,
Primary cutaneous anaplastic large-cell lymphoma,
Angioimmunoblastic T-cell lymphoma (AITL),
Cutaneous T-cell lymphoma (CTCL),
Mycosis fungoides (MF),
Sézary syndrome (SS),
Adult T-cell leukemia/lymphoma, and
Extranodal NK/T-cell lymphoma (ENK/TCL), nasal type.

Myeloproliferative disorders, in particular chronic myeloproliferative disorders (MPD), are rare blood cancers without clear causes. Examples include, without limitation, polycythemia vera, myelofibrosis, thrombocythemia, chronic neutrophilic leukemia, and eosinophilia.

In some embodiments, the cancer is AML. In some embodiments, the AML patient has a mutation in the fms related receptor tyrosine kinase 3 (FLT3) gene in the cancer cells. In some embodiments, the FLT3 mutation is a point mutation. In some embodiments, the FLT3 mutation is the internal tandem duplicate mutation (FLT3-ITD).

In some embodiments, the cancer is Waldenström macroglobulinemia (WM). In some embodiments, the WM patient has a mutation in the myeloid differentiation factor 88 (MyD88) gene. An example mutation is the missense mutation (L265P) that changes leucine at position 265 to proline in the protein.

It is also contemplated that the presently disclosed compounds and compositions can treat solid tumors. Non-limiting examples of solid tumors bladder cancer, liver cancer, colon cancer, rectal cancer, endometrial cancer, pancreatic cancer, small cell lung cancer, non-small cell lung cancer, breast cancer, urethral cancer, head and neck cancer, gastrointestinal cancer, stomach cancer, esophageal cancer, ovarian cancer, renal cancer, melanoma, prostate cancer and thyroid cancer.

The presently disclosed compounds and compositions may treat cancers that are normally challenging to treat. In some embodiments, the cancer is relapsed and/or refractory. In some embodiments, the cancer is advanced or metastatic.

Combination therapies are also provided, in some embodiments. As demonstrated in the experimental examples, Compound A synergized with poly ADP ribose polymerase (PARP) inhibitors (e.g., olaparib) and Bruton's tyrosine kinase (BTK) inhibitors (e.g., ibrutinib) in activating the apoptosis of the tested cancer cells. In some embodiments, therefore, the treatment of the present disclosure further entails administering to the cancer patient a PARP inhibitor and/or a BTK inhibitor. In some embodiments, the administration of Compound A and the PARP/BTK inhibitor is concurrent. In some embodiments, the administration of Compound A and the PARP/BTK inhibitor is sequential.

PARP inhibitors are pharmacological inhibitors of the enzyme poly ADP ribose polymerase (PARP). PARP is a family of proteins involved in the cellular processes including DNA repair, genomic stability, and programmed cell death. Examples of PARP inhibitors include, without limitation, olaparib, rucaparib, niraparib, talazoparib, veliparib, pamiparib, and iniparib.

Olaparib, for instance, has a chemical name of 4-[(3-[(4-cyclopropylcarbonyl)piperazin-1-yl]carbonyl)-4-fluorophenyl]methyl(2H)phthalazin-1-one. Its structure is shown below:

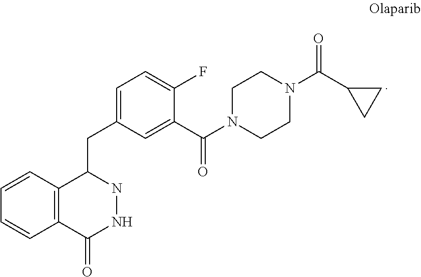 [SMILES: O=C1NNC(Cc2ccc(F)c(C(=O)N3CCN(C(=O)C4CC4)CC3)c2)c2ccccc21]

In some embodiments, the patient being treated with Compound A and the PARP inhibitor (e.g., olaparib) has a hematologic cancer. In some embodiments, the hematologic cancer is leukemia or lymphoma. In some embodiments, the hematologic cancer is AML, optionally with a FLT3 mutation, such as FLT3-ITD. In some embodiments, the hematologic cancer is WM, optionally with a L265P mutation in the MyD88 gene.

In some embodiments, Compound A is administered prior to the PARP inhibitor. In some embodiments, Compound A is administered after the PARP inhibitor. In some embodiments, they are administered at the same time, optionally as a combined dosage form. In some embodiments, Compound A and the PARP inhibitor are administered at a molar ratio of 1:20 to 20:1, 1:10 to 10:1, 1:8 to 8:1, 1:5 to 5:1, 1:4 to 4:1, 1:3 to 3:1, 1:2 to 2:1, 1:1.5 to 1.5:1, 1:1.2 to 1.2:1, 1:20 to 1:1, 1:20 to 1:2, 1:20 to 1:4, 1:20 to 1:5, 1:20 to 1:10, 1:1 to 1:20, 1:2 to 1:20, 1:4 to 1:20, 1:5 to 1:20, 1:10 to 1:20, 1:15 to 1:1, 1:15 to 1:2, 1:15 to 1:4, 1:15 to 1:5, 1:1 to 1:15, 1:2 to 1:15, 1:4 to 1:15, 1:5 to 1:15, 1:10 to 1:1, 1:10 to 1:2, 1:10 to 1:4, 1:10 to 1:5, 1:1 to 1:10, 1:2 to 1:10, 1:4 to 1:10, 1:5 to 1:10, 1:9 to 1:1, 1:9 to 1:2, 1:9 to 1:4, 1:9 to 1:5, 1:1 to 1:9, 1:2 to 1:9, 1:4 to 1:9, 1:5 to 1:9, 1:8 to 1:1, 1:8 to 1:2, 1:8 to 1:4, 1:8 to 1:5, 1:1 to 1:8, 1:2 to 1:8, 1:4 to 1:8, 1:5 to 1:8, 1:5 to 1:1, 1:5 to 1:2, 1:5 to 1:4, 1:1 to 1:5, 1:2 to 1:5, 1:4 to 1:5, 1:4 to 1:1, 1:4 to 1:2, 1:1 to 1:4, 1:2 to 1:4, 1:3 to 1:1, 1:3 to 1:2, 1:1 to 1:3, or 1:2 to 1:3, without limitation.

Bruton's tyrosine kinase (BTK), also known as tyrosine-protein kinase BTK, is a tyrosine kinase. BTK plays an important role in B cell development as it is required for transmitting signals from the pre-B cell receptor that forms after successful immunoglobulin heavy chain rearrangement. It also has a role in mast cell activation through the high-affinity IgE receptor.

A BTK inhibitor is a biological or chemical agent that binds to BTK and inhibits its activity. Non-limiting examples include ibrutinib, acalabrutinib, zanubrutinib, tirabrutinib, tolebrutinib, evobrutinib, fenebrutinib, pirtobrutinib, and spebrutinib.

Ibrutinib, for instance, has the chemical name of 1-[(3R)-3-[4-Amino-3-(4-phenoxyphenyl)-1H-pyrazolo[3,4-d]pyrimidin-1-yl]piperidin-1-yl]prop-2-en-1-one, and the following structure:

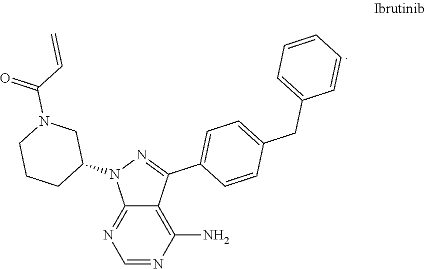 [SMILES: C=CC(=O)N1CCC[C@@H](n2nc(-c3ccc(Cc4ccccc4)cc3)c3c(N)ncnc32)C1]

In some embodiments, the patient being treated with Compound A and the BTK inhibitor (e.g., ibrutinib) has a hematologic cancer. In some embodiments, the hematologic cancer is leukemia or lymphoma. In some embodiments, the hematologic cancer is AML, optionally with a FLT3 mutation, such as FLT3-ITD. In some embodiments, the hematologic cancer is WM, optionally with a L265P mutation in the MyD88 gene.

In some embodiments, Compound A is administered prior to the BTK inhibitor. In some embodiments, Compound A is administered after the BTK inhibitor. In some embodiments, they are administered at the same time, optionally as a combined dosage form. In some embodiments, Compound A and the BTK inhibitor are administered at a molar ratio of 1:20 to 20:1, 1:10 to 10:1, 1:8 to 8:1, 1:5 to 5:1, 1:4 to 4:1, 1:3 to 3:1, 1:2 to 2:1, 1:1.5 to 1.5:1, 1:1.2 to 1.2:1, 1:20 to 1:1, 1:20 to 1:2, 1:20 to 1:4, 1:20 to 1:5, 1:20 to 1:10, 1:1 to 1:20, 1:2 to 1:20, 1:4 to 1:20, 1:5 to 1:20, 1:10 to 1:20, 1:15 to 1:1, 1:15 to 1:2, 1:15 to 1:4, 1:15 to 1:5, 1:1 to 1:15, 1:2 to 1:15, 1:4 to 1:15, 1:5 to 1:15, 1:10 to 1:1, 1:10 to 1:2, 1:10 to 1:4, 1:10 to 1:5, 1:1 to 1:10, 1:2 to 1:10, 1:4 to 1:10, 1:5 to 1:10, 1:9 to 1:1, 1:9 to 1:2, 1:9 to 1:4, 1:9 to 1:5, 1:1 to 1:9, 1:2 to 1:9, 1:4 to 1:9, 1:5 to 1:9, 1:8 to 1:1, 1:8 to 1:2, 1:8 to 1:4, 1:8 to 1:5, 1:1 to 1:8, 1:2 to 1:8, 1:4 to 1:8, 1:5 to 1:8, 1:5 to 1:1, 1:5 to 1:2, 1:5 to 1:4, 1:1 to 1:5, 1:2 to 1:5, 1:4 to 1:5, 1:4 to 1:1, 1:4 to 1:2, 1:1 to 1:4, 1:2 to 1:4, 1:3 to 1:1, 1:3 to 1:2, 1:1 to 1:3, or 1:2 to 1:3, without limitation.

In some embodiments, the treatment entails administration of all of Compound A, a PARP inhibitor and a BTK inhibitor.

### Pharmaceutical Compositions and Modes of Administration

Compounds provided herein are usually administered in the form of pharmaceutical compositions. Thus, provided herein are also pharmaceutical compositions that contain one or more of the compounds described herein or a pharmaceutically acceptable salt, tautomer, stereoisomer, mixture of stereoisomers, prodrug, or deuterated analog thereof and one or more pharmaceutically acceptable vehicles selected from carriers, adjuvants and excipients. Suitable pharmaceutically acceptable vehicles may include, for example, inert solid diluents and fillers, diluents, including sterile aqueous solution and various organic solvents, permeation enhancers, solubilizers and adjuvants. Such compositions are prepared in a manner well known in the pharmaceutical art. See, e.g., Remington's Pharmaceutical Sciences, Mace Publishing Co., Philadelphia, Pa. 17th Ed. (1985); and Modern Pharmaceutics, Marcel Dekker, Inc. 3rd Ed. (G. S. Banker & C. T. Rhodes, Eds.).

In some embodiments, the pharmaceutical composition includes Compound A or a salt thereof. In some embodiments, the pharmaceutical composition is a combination dosage form that further includes a PARP inhibitor and/or a BTK inhibitor.

Examples of PARP inhibitors include, without limitation, olaparib, rucaparib, niraparib, talazoparib, veliparib, pamiparib, and iniparib. Non-limiting examples include ibrutinib, acalabrutinib, zanubrutinib, tirabrutinib, tolebrutinib, evobrutinib, fenebrutinib, pirtobrutinib, and spebrutinib.

In some embodiments, Compound A and the PARP inhibitor are present in the combination dosage form at a molar ratio of 1:20 to 20:1, 1:10 to 10:1, 1:8 to 8:1, 1:5 to 5:1, 1:4 to 4:1, 1:3 to 3:1, 1:2 to 2:1, 1:1.5 to 1.5:1, 1:1.2 to 1.2:1, 1:20 to 1:1, 1:20 to 1:2, 1:20 to 1:4, 1:20 to 1:5, 1:20 to 1:10, 1:1 to 1:20, 1:2 to 1:20, 1:4 to 1:20, 1:5 to 1:20, 1:10 to 1:20, 1:15 to 1:1, 1:15 to 1:2, 1:15 to 1:4, 1:15 to 1:5, 1:1 to 1:15, 1:2 to 1:15, 1:4 to 1:15, 1:5 to 1:15, 1:10 to 1:1, 1:10 to 1:2, 1:10 to 1:4, 1:10 to 1:5, 1:1 to 1:10, 1:2 to 1:10, 1:4 to 1:10, 1:5 to 1:10, 1:9 to 1:1, 1:9 to 1:2, 1:9 to 1:4, 1:9 to 1:5, 1:1 to 1:9, 1:2 to 1:9, 1:4 to 1:9, 1:5 to 1:9, 1:8 to 1:1, 1:8 to 1:2, 1:8 to 1:4, 1:8 to 1:5, 1:1 to 1:8, 1:2 to 1:8, 1:4 to 1:8, 1:5 to 1:8, 1:5 to 1:1, 1:5 to 1:2, 1:5 to 1:4, 1:1 to 1:5, 1:2 to 1:5, 1:4 to 1:5, 1:4 to 1:1, 1:4 to 1:2, 1:1 to 1:4, 1:2 to 1:4, 1:3 to 1:1, 1:3 to 1:2, 1:1 to 1:3, or 1:2 to 1:3, without limitation.

In some embodiments, Compound A and the BTK inhibitor are present in the combination dosage form at a molar ratio of 1:20 to 20:1, 1:10 to 10:1, 1:8 to 8:1, 1:5 to 5:1, 1:4 to 4:1, 1:3 to 3:1, 1:2 to 2:1, 1:1.5 to 1.5:1, 1:1.2 to 1.2:1, 1:20 to 1:1, 1:20 to 1:2, 1:20 to 1:4, 1:20 to 1:5, 1:20 to 1:10, 1:1 to 1:20, 1:2 to 1:20, 1:4 to 1:20, 1:5 to 1:20, 1:10 to 1:20, 1:15 to 1:1, 1:15 to 1:2, 1:15 to 1:4, 1:15 to 1:5, 1:1 to 1:15, 1:2 to 1:15, 1:4 to 1:15, 1:5 to 1:15, 1:10 to 1:1, 1:10 to 1:2, 1:10 to 1:4, 1:10 to 1:5, 1:1 to 1:10, 1:2 to 1:10, 1:4 to 1:10, 1:5 to 1:10, 1:9 to 1:1, 1:9 to 1:2, 1:9 to 1:4, 1:9 to 1:5, 1:1 to 1:9, 1:2 to 1:9, 1:4 to 1:9, 1:5 to 1:9, 1:8 to 1:1, 1:8 to 1:2, 1:8 to 1:4, 1:8 to 1:5, 1:1 to 1:8, 1:2 to 1:8, 1:4 to 1:8, 1:5 to 1:8, 1:5 to 1:1, 1:5 to 1:2, 1:5 to 1:4, 1:1 to 1:5, 1:2 to 1:5, 1:4 to 1:5, 1:4 to 1:1, 1:4 to 1:2, 1:1 to 1:4, 1:2 to 1:4, 1:3 to 1:1, 1:3 to 1:2, 1:1 to 1:3, or 1:2 to 1:3, without limitation.

The pharmaceutical compositions may be administered in either single or multiple doses. The pharmaceutical composition may be administered by various methods including, for example, rectal, buccal, intranasal and transdermal routes. In certain embodiments, the pharmaceutical composition may be administered by intra-arterial injection, intravenously, intraperitoneally, parenterally, intramuscularly, subcutaneously, orally, topically, or as an inhalant.

One mode for administration is parenteral, for example, by injection. The forms in which the pharmaceutical compositions described herein may be incorporated for administration by injection include, for example, aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Oral administration may be another route for administration of the compounds described herein. Administration may be via, for example, capsule or enteric coated tablets. In making the pharmaceutical compositions that include at least one compound described herein or a pharmaceutically acceptable salt, tautomer, stereoisomer, mixture of stereoisomers, prodrug, or deuterated analog thereof, the active ingredient is usually diluted by an excipient and/or enclosed within such a carrier that can be in the form of a capsule, sachet, paper or other container. When the excipient serves as a diluent, it can be in the form of a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include lubricating agents such as talc, magnesium stearate, and mineral oil; wetting agents; emulsifying and suspending agents; preserving agents such as methyl and propylhydroxy-benzoates; sweetening agents; and flavoring agents.

The compositions that include at least one compound described herein or a pharmaceutically acceptable salt, tautomer, stereoisomer, mixture of stereoisomers, prodrug, or deuterated analog thereof can be formulated so as to provide quick, sustained or delayed release of the active ingredient after administration to the subject by employing procedures known in the art. Controlled release drug delivery systems for oral administration include osmotic pump systems and dissolutional systems containing polymer-coated reservoirs or drug-polymer matrix formulations. Examples of controlled release systems are given in U.S. Pat. Nos. 3,845,770; 4,326,525; 4,902,514; and 5,616,345. Another formulation for use in the methods disclosed herein employ transdermal delivery devices (“patches”). Such transdermal patches may be used to provide continuous or discontinuous infusion of the compounds described herein in controlled amounts. The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

For preparing solid compositions such as tablets, the principal active ingredient may be mixed with a pharmaceutical excipient to form a solid preformulation composition containing a homogeneous mixture of a compound described herein or a pharmaceutically acceptable salt, tautomer, stereoisomer, mixture of stereoisomers, prodrug, or deuterated analog thereof. When referring to these preformulation compositions as homogeneous, the active ingredient may be dispersed evenly throughout the composition so that the composition may be readily subdivided into equally effective unit dosage forms such as tablets, pills and capsules.

The tablets or pills of the compounds described herein may be coated or otherwise compounded to provide a dosage form affording the advantage of prolonged action, or to protect from the acid conditions of the stomach. For example, the tablet or pill can include an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, acetyl alcohol, and cellulose acetate.

Compositions for inhalation or insufflation may include solutions and suspensions in pharmaceutically acceptable, aqueous, or organic solvents, or mixtures thereof, and powders. The liquid or solid compositions may contain suitable pharmaceutically acceptable excipients as described herein. In some embodiments, the compositions are administered by the oral or nasal respiratory route for local or systemic effect. In other embodiments, compositions in pharmaceutically acceptable solvents may be nebulized by use of inert gases. Nebulized solutions may be inhaled directly from the nebulizing device, or the nebulizing device may be attached to a facemask tent, or intermittent positive pressure breathing machine. Solution, suspension, or powder compositions may be administered, preferably orally or nasally, from devices that deliver the formulation in an appropriate manner.

### Dosing

The specific dose level of a compound of the present application for any particular subject will depend upon a variety of factors including the activity of the specific compound employed, the age, body weight, general health, sex, diet, time of administration, route of administration, and rate of excretion, drug combination and the severity of the particular disease in the subject undergoing therapy. For example, a dosage may be expressed as a number of milligrams of a compound described herein per kilogram of the subject's body weight (mg/kg). Dosages of between about 0.1 and 150 mg/kg may be appropriate. In some embodiments, about 0.1 and 100 mg/kg may be appropriate. In other embodiments a dosage of between 0.5 and 60 mg/kg may be appropriate. Normalizing according to the subject's body weight is particularly useful when adjusting dosages between subjects of widely disparate size, such as occurs when using the drug in both children and adult humans or when converting an effective dosage in a non-human subject such as dog to a dosage suitable for a human subject.

The daily dosage may also be described as a total amount of a compound described herein administered per dose or per day. Daily dosage of Compound A may be between about 1 mg and 4,000 mg, between about 2,000 to 4,000 mg/day, between about 1 to 2,000 mg/day, between about 1 to 1,000 mg/day, between about 10 to 500 mg/day, between about 20 to 500 mg/day, between about 50 to 300 mg/day, between about 75 to 200 mg/day, or between about 15 to 150 mg/day.

When administered orally, the total daily dosage for a human subject may be between 1 mg and 1,000 mg, between about 1,000-2,000 mg/day, between about 10-500 mg/day, between about 50-300 mg/day, between about 75-200 mg/day, or between about 100-150 mg/day.

The compounds of the present application or the compositions thereof may be administered once, twice, three, or four times daily, using any suitable mode described above. Also, administration or treatment with the compounds may be continued for a number of days; for example, commonly treatment would continue for at least 7 days, 14 days, or 28 days, for one cycle of treatment. Treatment cycles are well known in cancer chemotherapy, and are frequently alternated with resting periods of about 1 to 28 days, commonly about 7 days or about 14 days, between cycles. The treatment cycles, in other embodiments, may also be continuous.

In a particular embodiment, the method comprises administering to the subject an initial daily dose of about 1 to 800 mg of a compound described herein and increasing the dose by increments until clinical efficacy is achieved. Increments of about 5, 10, 25, 50, or 100 mg can be used to increase the dose. The dosage can be increased daily, every other day, twice per week, or once per week.

### EXAMPLES

The following examples are included to demonstrate specific embodiments of the disclosure. It should be appreciated by those of skill in the art that the techniques disclosed in the examples which follow represent techniques to function well in the practice of the disclosure, and thus can be considered to constitute specific modes for its practice. However, those of skill in the art should, in light of the present disclosure, appreciate that many changes can be made in the specific embodiments which are disclosed and still obtain a like or similar result without departing from the spirit and scope of the disclosure.

### Example 1. Treatment of FLT3 Mutated Leukemia

This example tested the efficacy of Compound A in treating leukemia with FLT3 (fms related receptor tyrosine kinase 3) mutations.

Around 30% of the acute myeloid leukemia (AML) patient present a FLT3 mutation. FLT3 mutations can be divided in 2 groups. Punctual mutations represent 5% and internal tandem duplicates represent around 25% (FLT3-ITD). AML with FLT3-ITD mutations lead to significantly poor prognosis often requiring allogenic stem cell transplant.

In this example, MOLM-13 cells (adult acute myeloid leukemia) expressing the FLT3-ITD mutation were used to test Compound A to decrease their cell proliferation and increase their apoptosis level. Midostaurin (mido), an inhibitor of FLT3, was used as a positive control.

MOLM-13 cells were cultured in RPMI1640, red phenol free, in presence of 2, 10 and 25% of fetal bovine serum (FBS). 25% FBS simulates the level of serum present in the blood. Adipose stromal cells (ASC) and human fibroblast primary cells were used as reference controls. Compound A, at various concentrations (0.05 to 100 μM), was added to the cells and incubated for 24 h, with or without the co-treatment with olaparib (a PARP inhibitor, Ola). Olaparib or ibrutinib (a BTK inhibitor) alone was also tested with these cells.

### Results

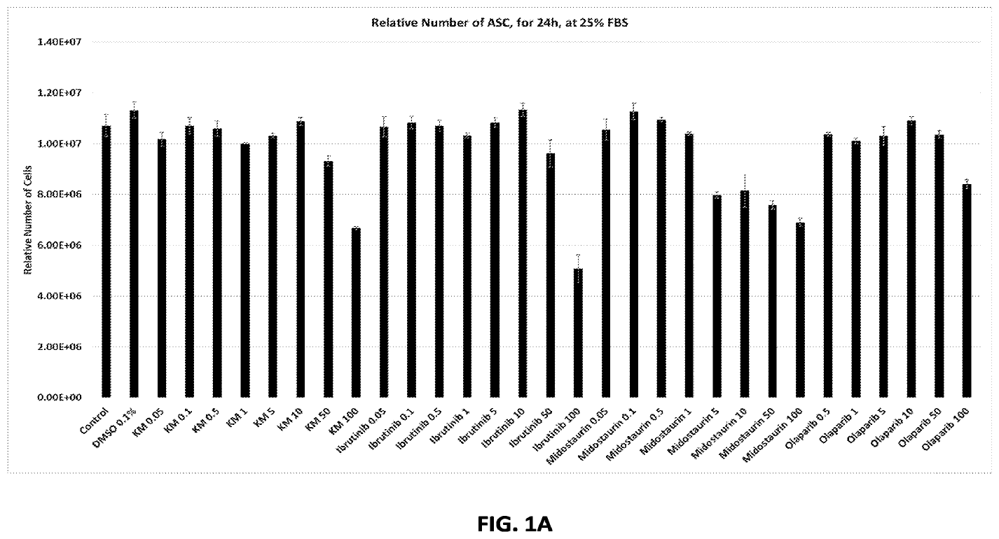

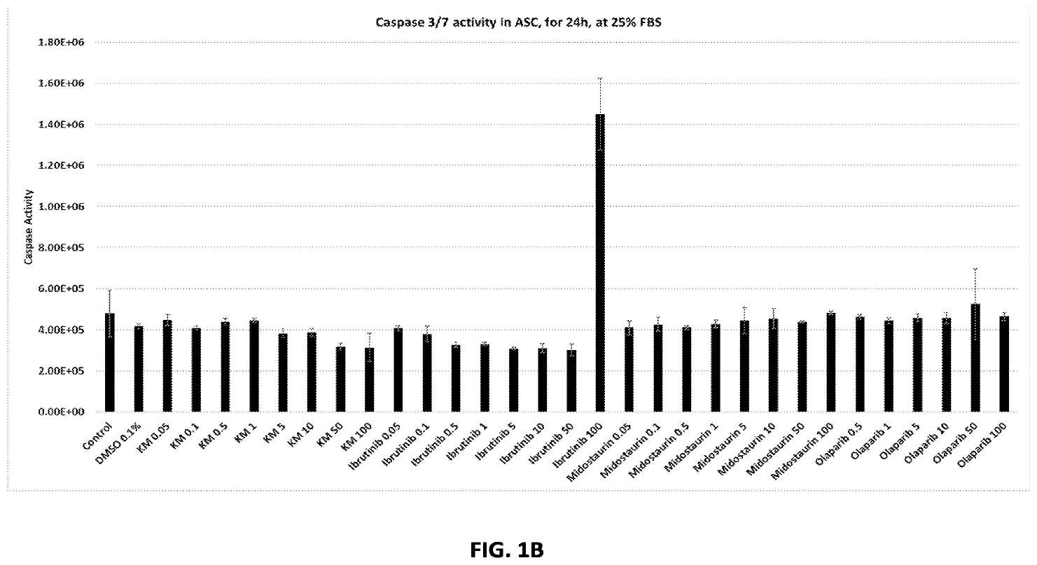

The testing results from the ASC cells are presented in FIG. 1A-B. The treatment with Compound A (labeled as “KM” in figures), at 50 and 100 μM but not at lower concentrations, resulted in slower cell number increase of human ASC as compared to vehicle control, but did not change the activity of caspase 3 or 7. This result indicates that Compound A inhibited cell growth but did not induce apoptosis. Like Compound A, olaparib also inhibited ASC growth at a higher concentration (100 μM) and did not induce apoptosis. Ibrutinib, by contrast, induced apoptosis (i.e., increased caspase 3/7 activities) of ASC at 100 PM, but not at lower concentrations. The induction of apoptosis by ibrutinib at this concentration also led to a decrease of ASC number as compared to vehicle control. As the positive control, midostaurin inhibited cell growth at all concentrations from 5 μM to 100 μM, and did not activate caspase 3/7.

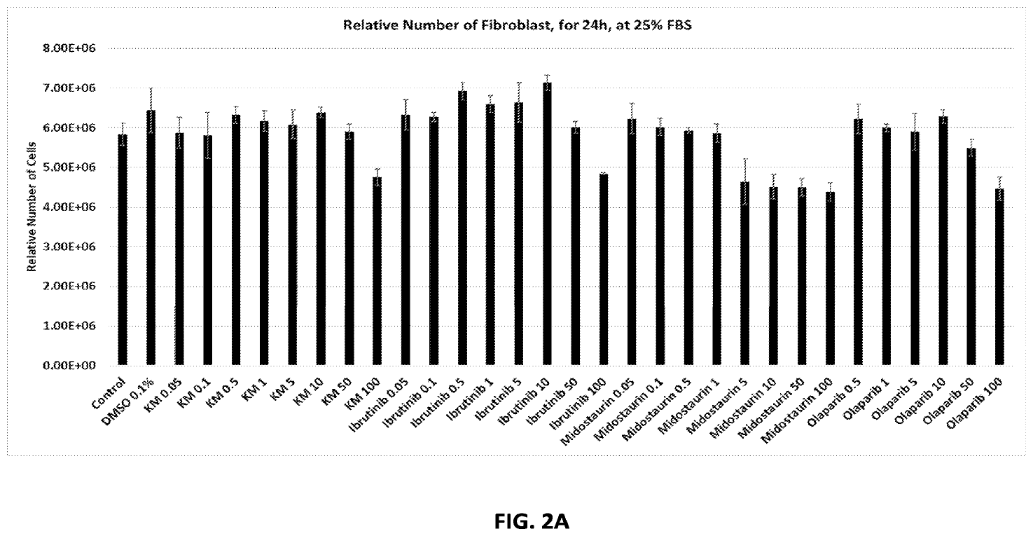

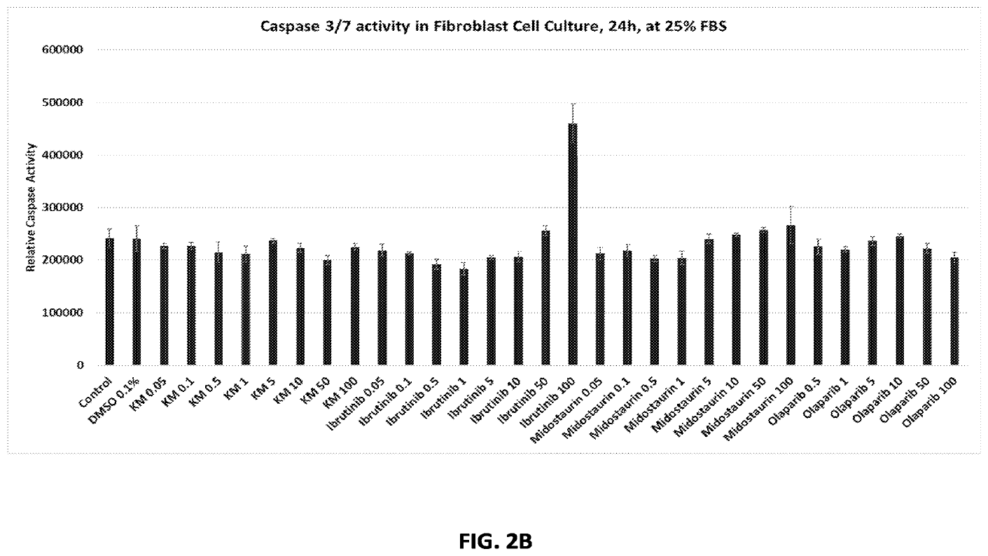

For human fibroblast primary cells (see results in FIG. 2A-B), the treatment with Compound A also resulted in slower cell growth at 100 μM (but not at lower concentrations). No apoptosis of the fibroblasts was observed with the Compound A treatment. Similar observations were made for olaparib at 50 and 100 μM. Again, at 100 μM, ibrutinib increased the caspase 3/7 activities in the fibroblasts.

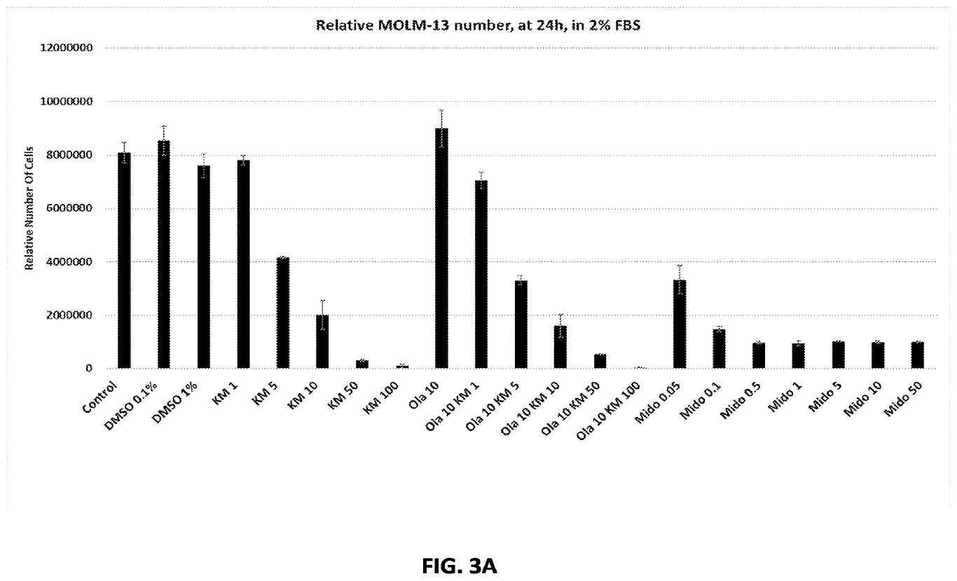

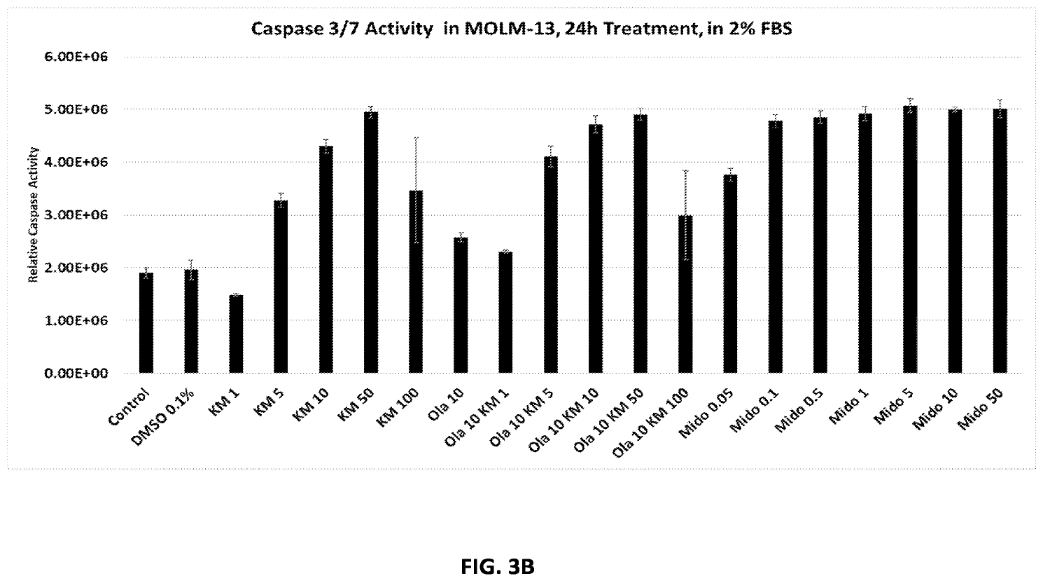

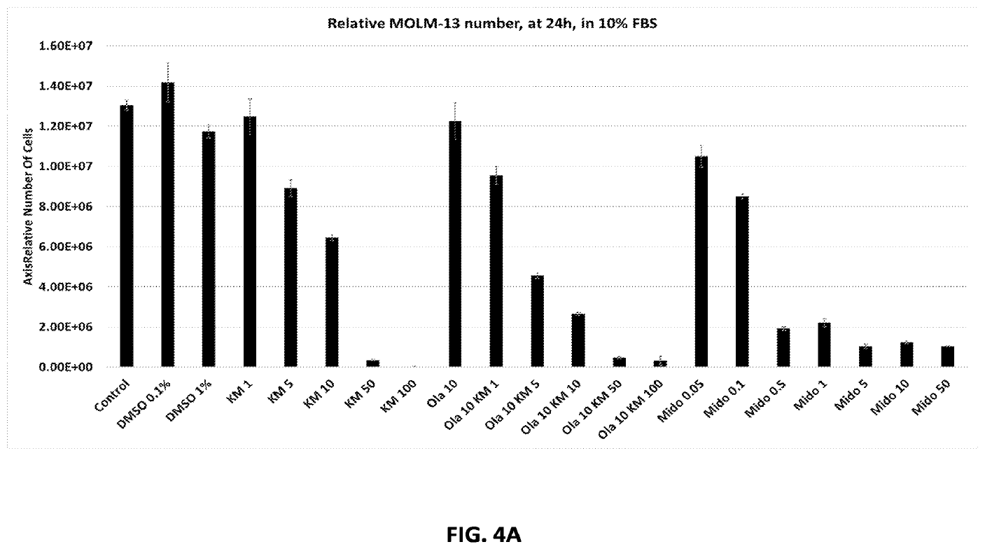

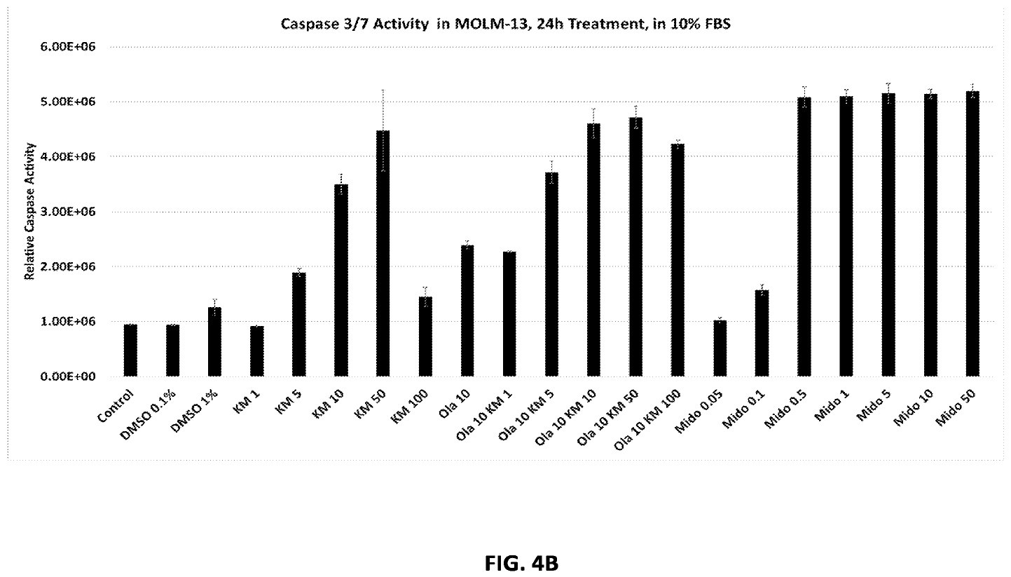

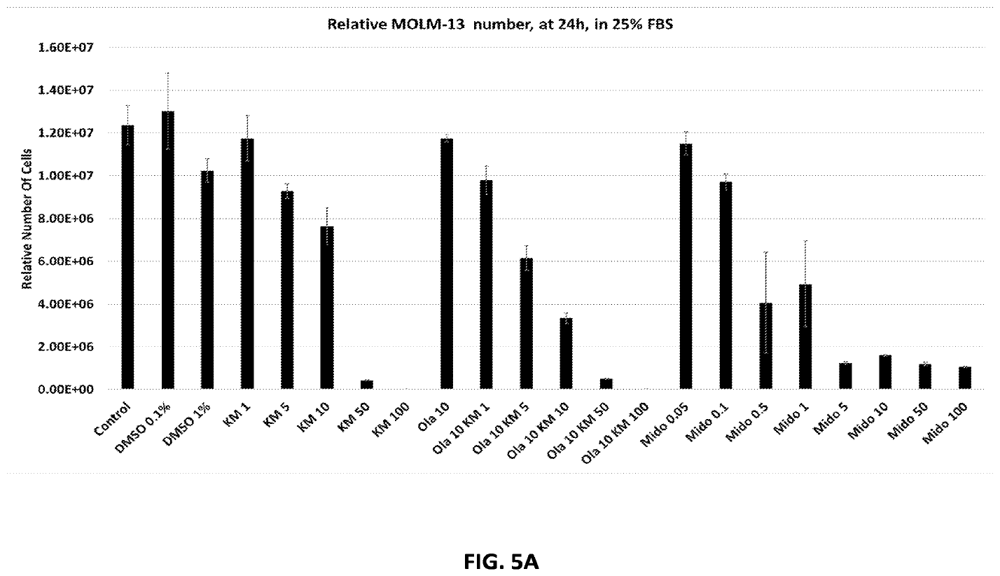

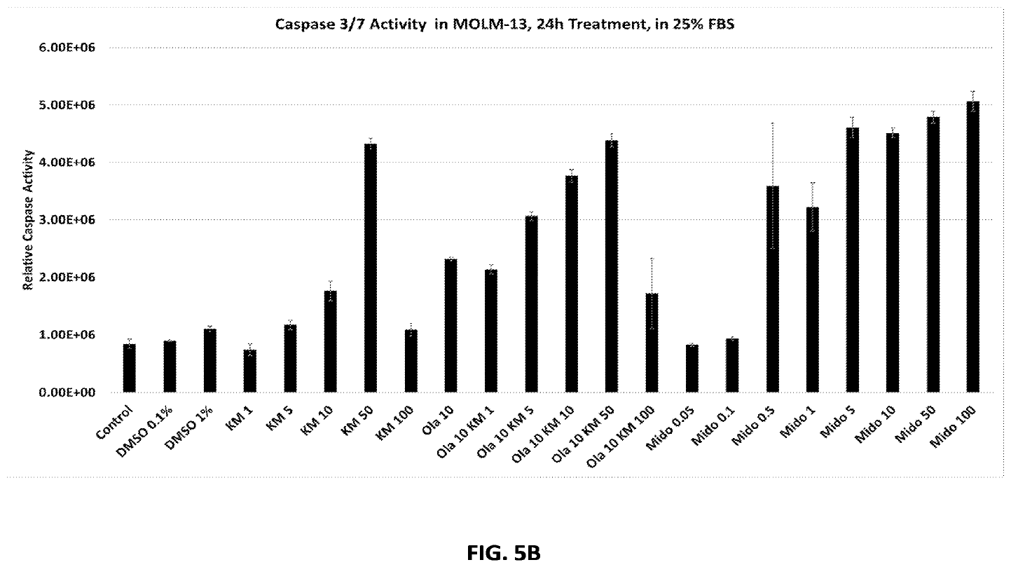

The impacts of Compound A on the MOLM-13 cells were quite different. As shown in FIG. 3-4, the numbers of MOLM-13 cells decreased (in 2% or 10% FBS), in a dose-dependent manner following the treatment with Compound A (1 to 100 μM). The level of caspase 3/7 activity was elevated, also in a dose-dependent manner, which is consistent with the decreased number of cells. MOLM-13 cells also responded to the anti-cancer effects of Compound A at 25% FBS (FIG. 5A-B), indicating that it could work on patients.

Midostaurin, from 0.05 to 100 μM, reduced the numbers of cells and increased caspase 3/7 activity in the MOLM-13 cells, in a dose-dependent manner, in 2, 10 or 25% FBS (FIG. 3-5). In addition, at the lowest doses, midostaurin was more efficient than Compound A, but Compound A was more efficient at higher doses than midostaurin.

Olaparib alone, at 10 μM, did not decrease the numbers of MOLM-13 cells but increased the apoptotic levels in 2, 10 or 25% FBS (FIG. 3-5). More interestingly, a synergistic effect was observed between olaparib (10 μM) and lower concentrations of Compound A (from 1 to 50 μM), in 25% FBS.

In summary, this Example demonstrates that Compound A and olaparib synergistically decreased the levels of cell proliferation and increased the levels of apoptosis in MOLM-13 cells. Compound A, or in combination with olaparib, can be suitably used in the treatment of AML expressing FLT-ITD.

### Example 2. Treatment of Waldenström Macroglobulinemia Cells with Myd88 L265P

In this study, MWCL-1 cells (adult Waldenström macroglobulinemia cells) expressing the Myd88 L265P mutation were used to test different drugs for their effects on cell proliferation and apoptosis. Midostaurin (an inhibitor of FLT3, mido) was used as reference control.

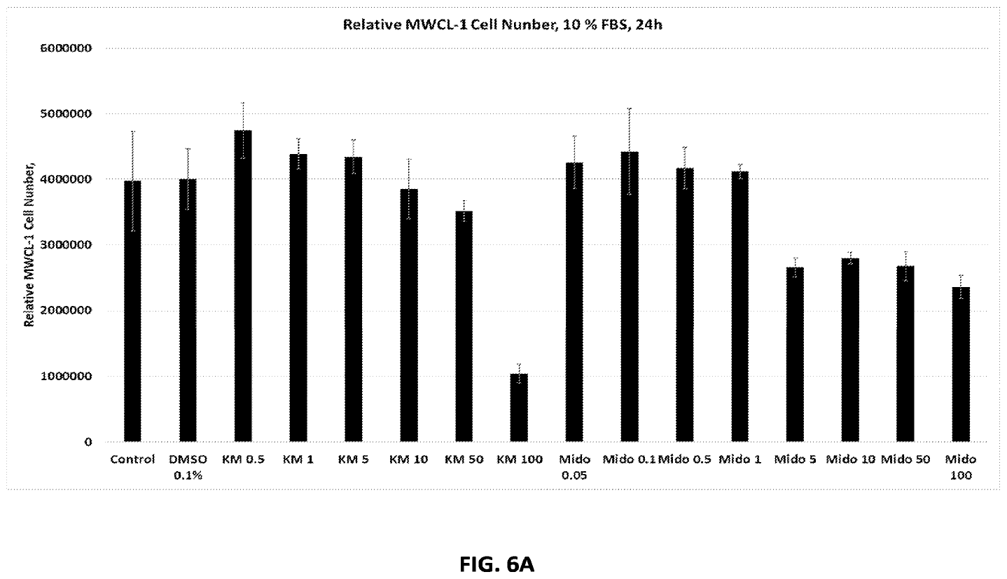

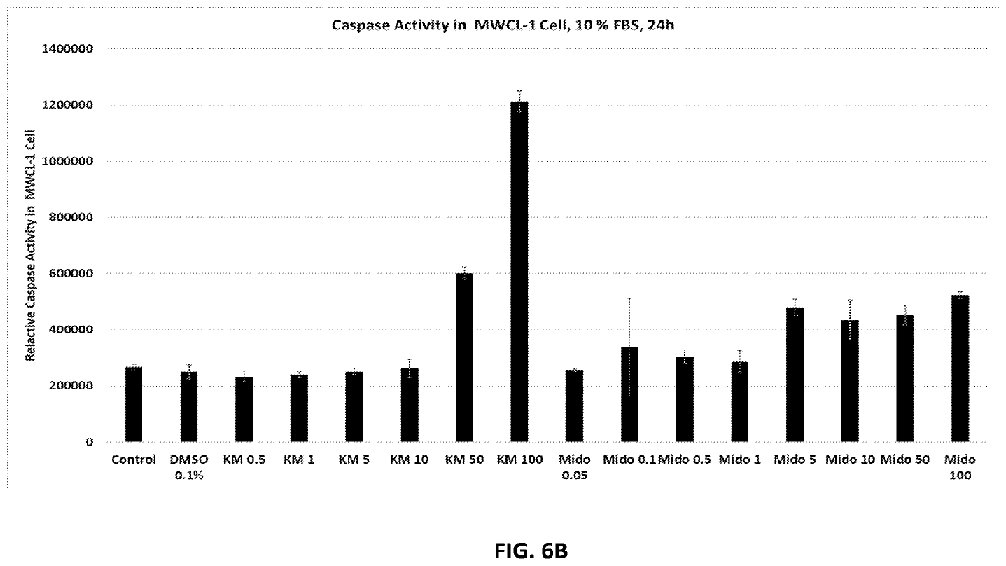

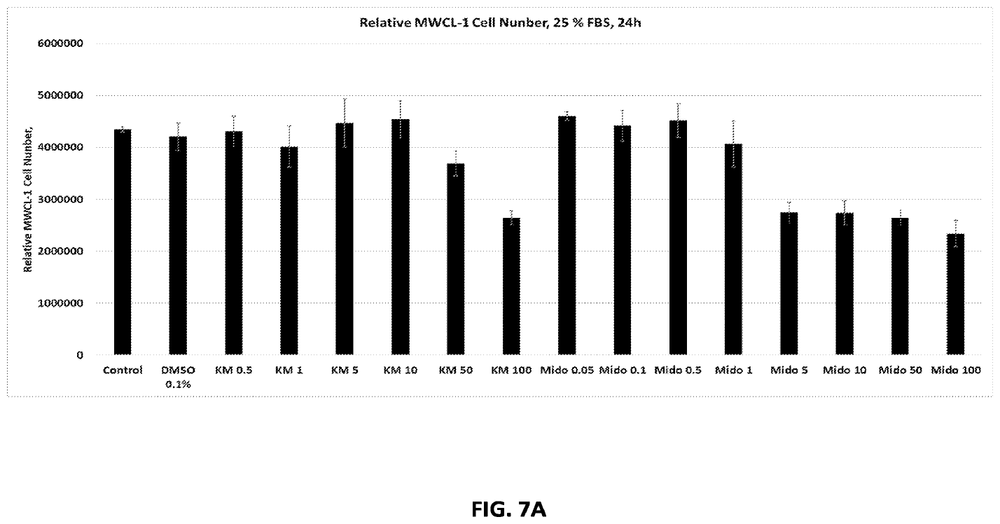

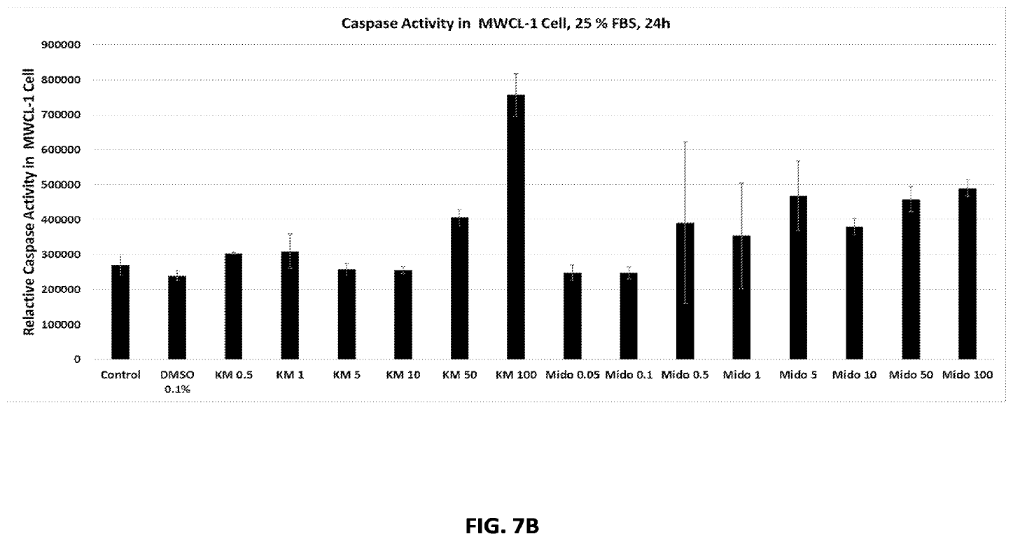

MWCL-1 cells were cultured in RPMI1640, in presence of 2, 10 and 25% of fetal bovine serum (FBS). The number of MWCL-1 cells decreased only at higher Compound A concentration (50 and 100 μM), during a 24 h treatment, at 2, 10 and 25% FBS (FIGS. 6A, 7A and 8A). Also, the levels of caspase 3/7 activity in the cells were elevated only at the higher concentrations (50 and 100 μM, FIGS. 6B, 7B and 8B), which is consistent with the decreased cell growth. It is noteworthy that MWCL-1 responded to the anti-cancer effects of Compound A in 25% FBS (FIG. 7A-B).

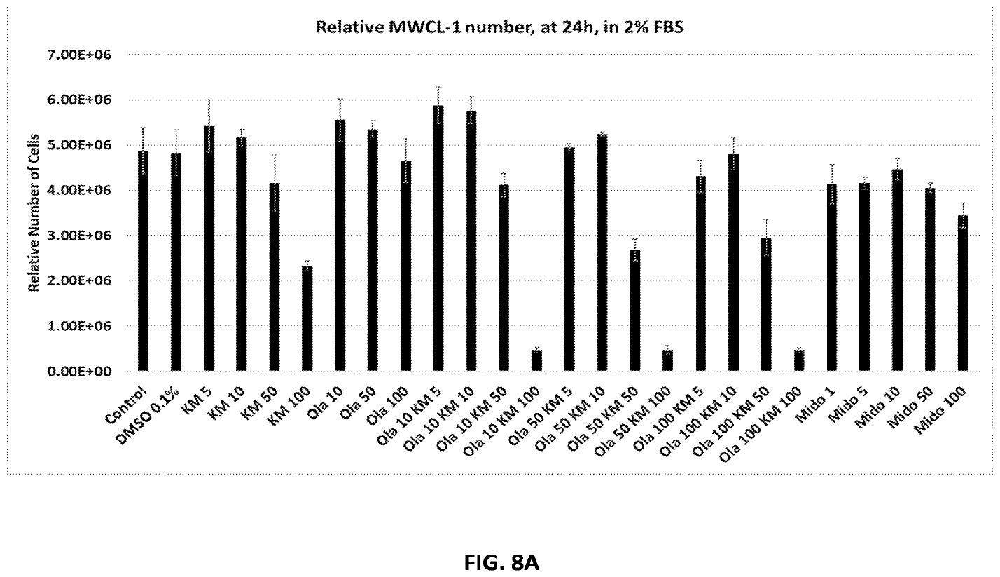

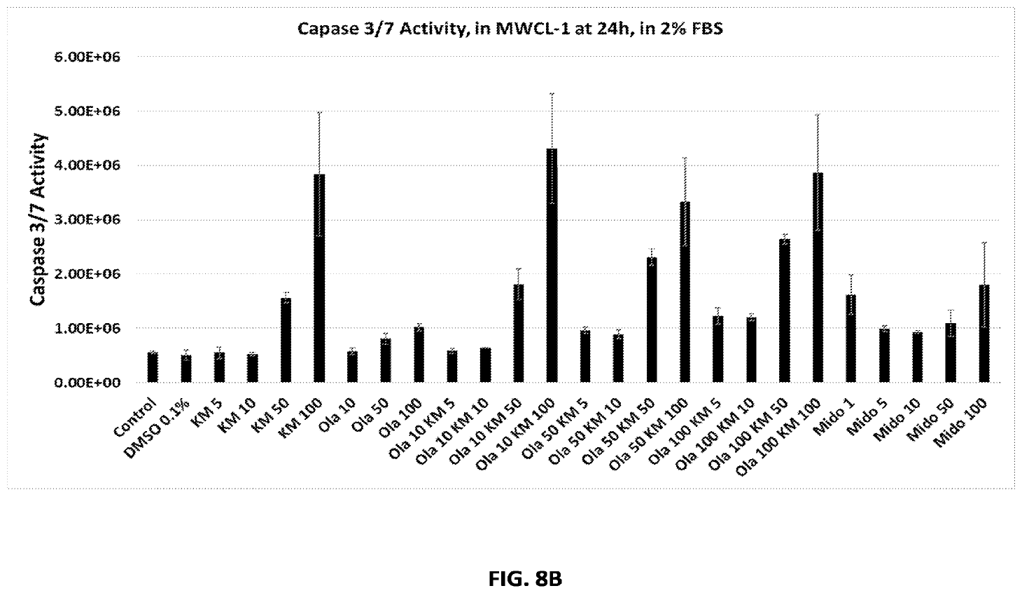

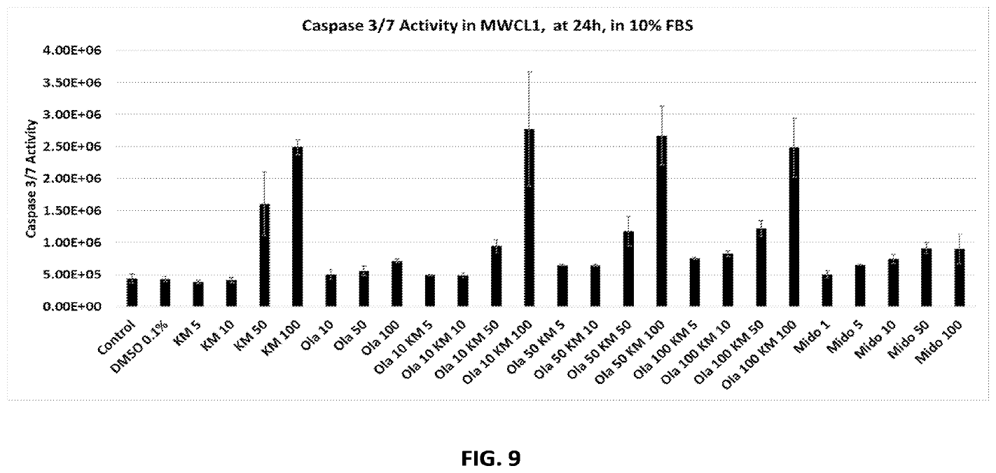

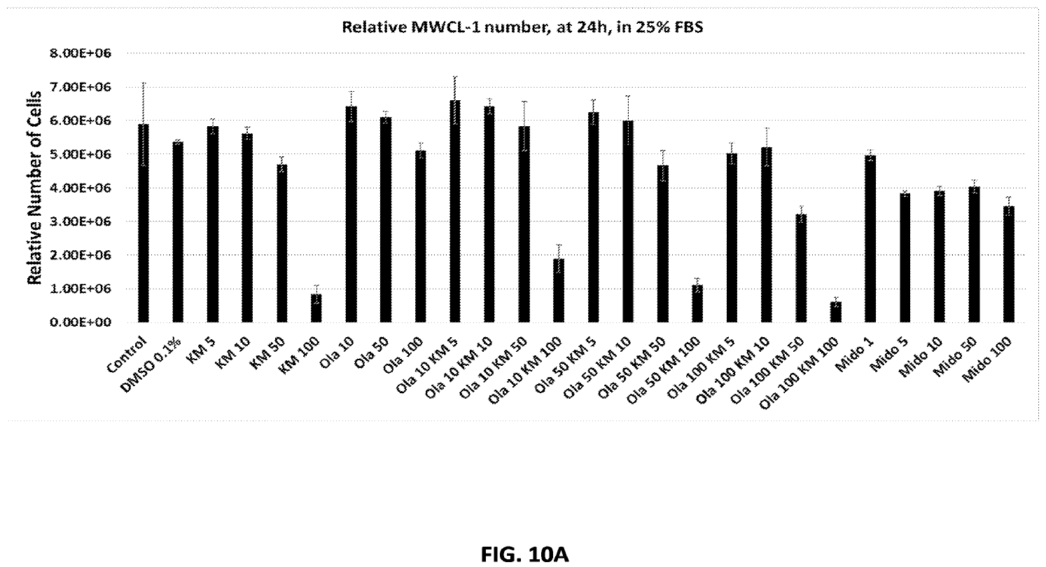

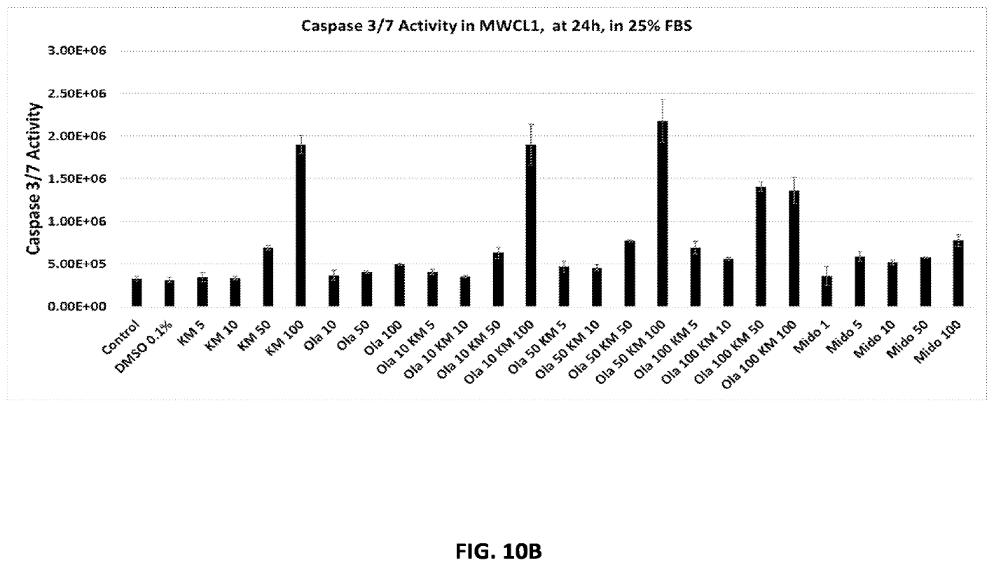

Midostaurin, from 0.05 to 100 μM, was reducing the number of MWCL-1 cells and increasing the caspase 3/7 activity in the cells, in a dose response manner, at 25% FBS (FIG. 7A-B). However, the data suggest that at the highest midostaurin dose (100 μM), Compound A was more efficient than midostaurin (100 μM). Olaparib at 50, 100 μM increased the caspase 3/7 activity, when MWCL-1 were cultured at 25% FBS (FIG. 8-10).

Interestingly, a synergistic effect was observed between olaparib at 100 μM and Compound A at 50 μM, between olaparib at 50 μM and Compound A at 50 μM (FIG. 8-10).

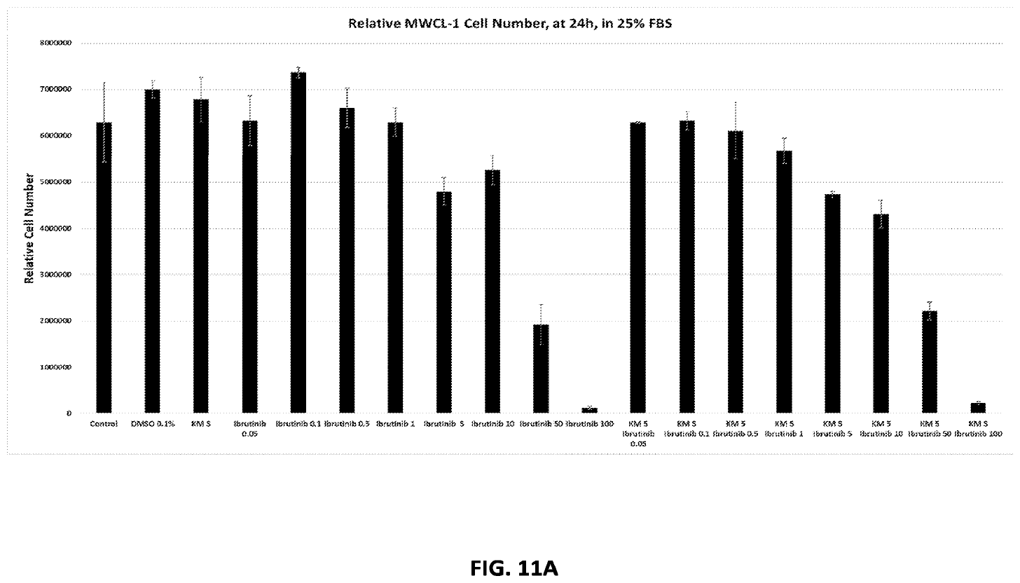

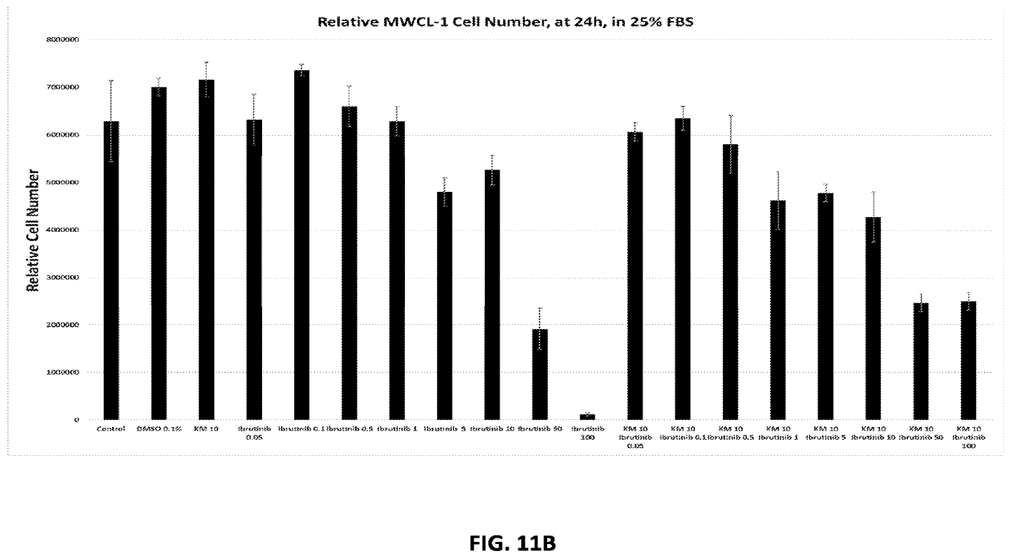

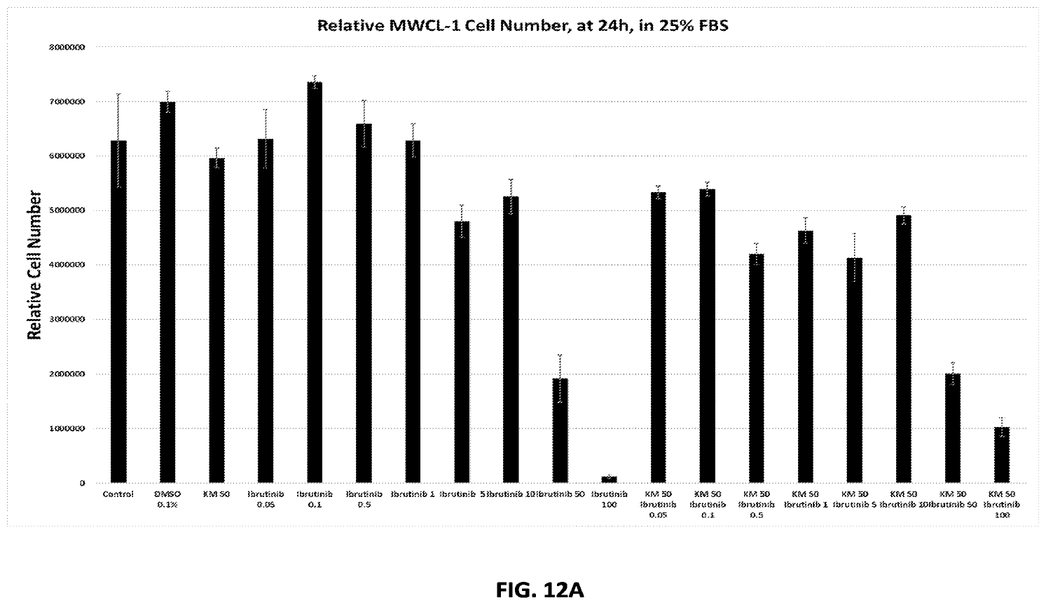

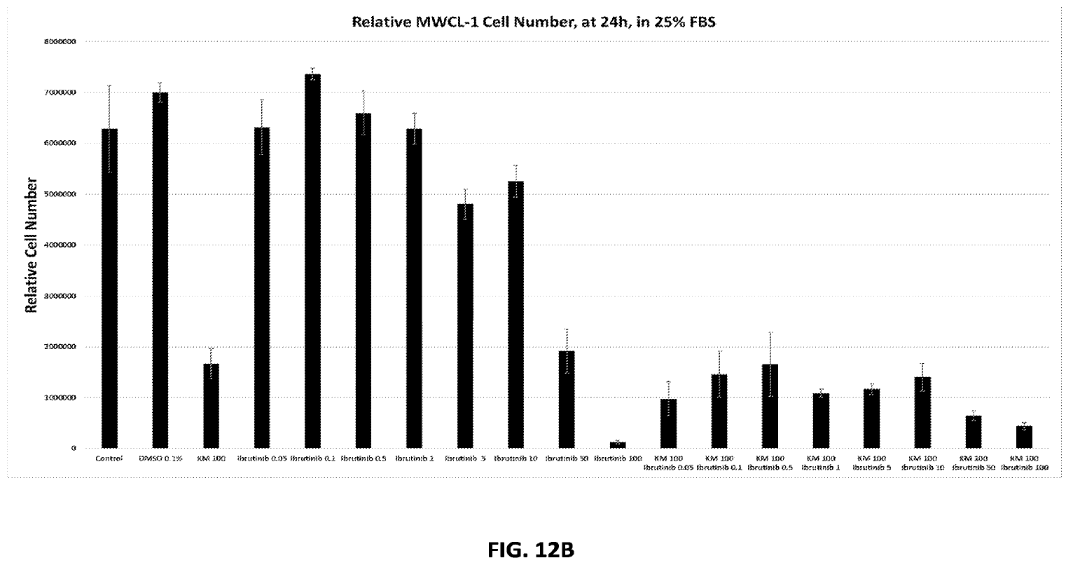

Further, as shown in FIG. 11-12, Compound A combined with ibrutinib had synergic effects on the number of MWCL-1 cells and the caspase 3/7 activity. In particular, at 25% FBS, Compound A (10 μM) combined with ibrutinib at 1 μM exhibited strong synergism in decreasing the number of MWCL-1 cells and increasing the caspase 3/7 activity. At 25% FBS, Compound A (50 μM) combined with ibrutinib at 0.05, 0.1, 0.5 and 1 μM also had such synergic effects, as well as between Compound A (100 μM) and ibrutinib at 1, 5 and 50 μM.

This example, therefore, demonstrates that Compound A alone or in combination with the olaparib or ibrutinib, significantly inhibited MWCL-1 cell proliferation and activated their apoptosis in 2 to 25% of FBS.

Unless otherwise defined, all technical and scientific terms used herein have the same meaning as commonly understood by one of ordinary skill in the art to which this invention belongs.

The inventions illustratively described herein may suitably be practiced in the absence of any element or elements, limitation or limitations, not specifically disclosed herein. Thus, for example, the terms “comprising”, “including,” “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

Thus, it should be understood that although the present invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

What is claimed is:

1. A method of treating cancer in a patient, comprising administering to the patient a compound of the structure of 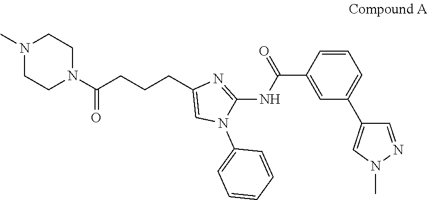 [SMILES: CN1CCN(C(=O)CCCc2cn(-c3ccccc3)c(NC(=O)c3cccc(-c4cnn(C)c4)c3)n2)CC1]
or a salt thereof.

2. The method of claim 1, wherein the salt is selected from the group consisting of hydrochloride salt, citrate salt, fumarate salt, (2R,3R)-2,3-dihydroxysuccinate salt, and succinate salt.

3. The method of claim 1, wherein the cancer is a hematologic cancer.

4. The method of claim 3, wherein the hematological cancer is selected from the group consisting of acute lymphocytic leukemia (ALL), acute myeloid leukemia (AML), chronic lymphocytic leukemia (CLL), chronic myeloid leukemia (CML), prolymphocytic leukemia (PLL), large granular lymphocytic (LGL) leukemia, hairy cell leukemia (HCL), and myelodysplastic syndrome (MDS).

5. The method of claim 4, wherein the hematologic cancer is AML.

6. The method of claim 5, wherein the patient has a fms related receptor tyrosine kinase 3 (FLT3) mutation.

7. The method of claim 6, wherein the FLT3 mutation is the internal tandem duplicate mutation (FLT3-ITD).

8. The method of claim 3, wherein the hematological cancer is selected from the group consisting of Hodgkin's lymphoma, diffuse large B-cell lymphoma (DLBCL), mantle cell lymphoma (MCL), lymphoblastic lymphoma, Burkitt lymphoma (BL), primary mediastinal (thymic) large B-cell lymphoma (PMBCL), transformed follicular and transformed mucosa-associated lymphoid tissue (MALT) lymphoma, high-grade B-cell lymphoma with double or triple hits (HBL), primary cutaneous DLBCL of the leg, primary DLBCL of the central nervous system, primary central nervous system (CNS) lymphoma, acquired immunodeficiency syndrome (AIDS)—associated lymphoma, follicular lymphoma (FL), marginal zone lymphoma (MZL), chronic lymphocytic leukemia/small-cell lymphocytic lymphoma (CLL/SLL), gastric mucosa-associated lymphoid tissue (MALT) lymphoma, lymphoplasmacytic lymphoma, Waldenström macroglobulinemia (WM), nodal marginal zone lymphoma (NMZL), splenic marginal zone lymphoma (SMZL), peripheral T-cell lymphoma (PTCL), systemic anaplastic large-cell lymphoma (ALCL), lymphoblastic lymphoma, hepatosplenic gamma/delta T-cell lymphoma, subcutaneous panniculitis-like T-cell lymphoma (SPTCL), enteropathy-type intestinal T-cell lymphoma, primary cutaneous anaplastic large-cell lymphoma, angioimmunoblastic T-cell lymphoma (AITL), cutaneous T-cell lymphoma (CTCL), mycosis fungoides (MF), Sézary syndrome (SS), adult T-cell leukemia/lymphoma, and extranodal NK/T-cell lymphoma (ENK/TCL).

9. The method of claim 8, wherein the hematologic cancer is Waldenström macroglobulinemia (WM).

10. The method of claim 9, wherein the patient has a L265P mutation in the myeloid differentiation factor 88 (MyD88) gene.

11. The method of claim 3, wherein the hematologic cancer is multiple myeloma.

12. The method of claim 3, wherein the hematologic cancer is selected from the group consisting of polycythemia vera, myelofibrosis, thrombocythemia, chronic neutrophilic leukemia, and eosinophilia.

13. The method of claim 1, wherein the cancer is a solid tumor.

14. The method of claim 13, wherein the cancer is selected from the group consisting of bladder cancer, liver cancer, colon cancer, rectal cancer, endometrial cancer, pancreatic cancer, small cell lung cancer, non-small cell lung cancer, breast cancer, urethral cancer, head and neck cancer, gastrointestinal cancer, stomach cancer, esophageal cancer, ovarian cancer, renal cancer, melanoma, prostate cancer and thyroid cancer.

15. The method of claim 1, wherein the cancer is relapsed and refractory.

16. The method of claim 1, further comprising administering to the patient a poly ADP ribose polymerase (PARP) inhibitor.

17. The method of claim 16, wherein the PARP inhibitor is selected from the group consisting of olaparib, rucaparib, niraparib, talazoparib, veliparib, pamiparib, and iniparib.

18. The method of claim 17, wherein the PARP inhibitor is olaparib.

19. The method of claim 1, further comprising administering to the patient a Bruton's tyrosine kinase (BTK) inhibitor.

20. (canceled)

22. A composition comprising a compound of the structure of 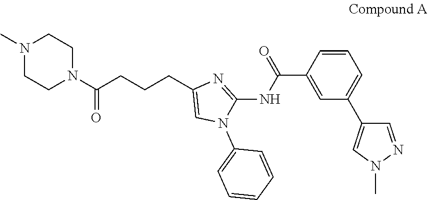 [SMILES: CN1CCN(C(=O)CCCc2cn(-c3ccccc3)c(NC(=O)c3cccc(-c4cnn(C)c4)c3)n2)CC1]
or a salt thereof and a PARP inhibitor or a BTK inhibitor.

23. (canceled)
